(19) United States
(12) Patent Application Publication
(10) Pub. No.: US 2016/0262652 A1
(43) Pub. Date: Sep. 15, 2016

(54) ELECTROMAGNETIC WAVE SENSOR AND METHOD OF GENERATING THE ELECTROMAGNETIC WAVE SENSOR

(71) Applicant: Electronics and Telecommunications Research Institute, Daejeon (KR)

(72) Inventors: Kwang Jae LEE, Daejeon (KR); Jang Yeol KIM, Daejeon (KR); Soon Ik JEON, Daejeon (KR)

(21) Appl. No.: 14/821,210

(22) Filed: Aug. 7, 2015

(30) Foreign Application Priority Data
Mar. 9, 2015 (KR) 10-2015-0032173

(51) Int. Cl. A61B 5/05; G01R 29/08

(52) U.S. Cl. CPC A61B 5/05; G01R 29/0814; G01R 29/0878

(57) ABSTRACT

Provided is an electromagnetic wave sensor including a thin metal plating layer to prevent an inflow of a fluid into the electromagnetic wave sensor and a method of generating the electromagnetic wave sensor, in which the electromagnetic wave sensor includes a waveguide including a conductor to sense an electromagnetic wave, a ceramic layer accommodated in the waveguide and including a dielectric to reduce a dielectric loss of the electromagnetic wave, and the thin metal plating layer disposed between the waveguide and the ceramic layer to prevent the inflow of the fluid into the electromagnetic wave sensor.

### CROSS-REFERENCE TO RELATED APPLICATION

This application claims priority to and the benefit of Korean Patent Application No. 10-2015-0032173, filed on Mar. 9, 2015, in the Korean Intellectual Property Office, the disclosure of which is incorporated herein by reference.

### BACKGROUND

1. Field of the Invention

The present invention relates to an electromagnetic wave sensor, and more particularly, to an electromagnetic wave sensor including a thin metal plating layer to prevent an inflow of a fluid into the electromagnetic wave sensor and a method of generating the electromagnetic wave sensor.

2. Description of the Related Art

An electromagnetic wave imaging apparatus for medical purposes may project an electromagnetic wave to a human body tissue and measure a scattered signal. Based on the measured signal and data obtained accordingly, the tissue may be examined and analyzed. Such an apparatus is widely used because a diagnosis and an examination may be conducted without incising or injuring a human body tissue. However, when a sensor is disposed in a free space, for example, in air, and an electromagnetic wave is projected to a human body tissue, a large number of reflected waves may be generated from a surface of the tissue. According to an electromagnetic theory, a human body tissue may be equivalent to a dielectric having a high relative permittivity, and a surface on which air having a low relative permittivity is in contact with the tissue having a high relative permittivity may generate a large number of reflections.

Thus, the electromagnetic wave imaging apparatus for medical purposes may use a sensing method of filling, with a fluid, a gap between an opening of the sensor and the tissue so that an electromagnetic wave signal including information is transferred to the tissue without hindrance. However, in a case of using such a sensing method, a fluid may permeate through an assembly gap of the electromagnetic wave sensor over time. The inflow of the fluid may lead to a loss of electromagnetic phenomena in the sensor and deterioration in electrical performances of the sensor. Thus, designing existing sensors to be in a completely sealed structure may be necessary. However, the designing may require a considerably high level of technical ability, and increase production costs and also additional costs for repair and maintenance for maintaining the sealed state for a long period of time.

Accordingly, there is a desire for a method of preventing permeation of a fluid and deterioration in a sensing performance without using expensive sealing technology.

### PATENT DOCUMENTS

Patent Document No. 001: Korean Patent Application No. 10-1993-0008233 filed on May 13, 1993, and entitled “MOLDED WAVEGUIDE COMPONENTS ELECTROLESS-PLATED THERMOPLASTIC MEMBERS”

Patent Document No. 002: Korean Patent Application No. 10-2002-0013581 filed on Mar. 13, 2002, and entitled “THE WAVEGUIDE SLOT ANTENNA AND MANUFACTURING METHOD THEREOF”

Patent Document No. 003: Korean Patent Application No. 10-2011-7020919 filed on Mar. 31, 2010, and entitled “WAVEGUIDE”

### SUMMARY

According to an aspect of the present invention, there is provided an electromagnetic wave sensor including a waveguide including a conductor to sense an electromagnetic wave, a ceramic layer accommodated in the waveguide and including a dielectric to reduce a dielectric loss of the electromagnetic wave, and a thin metal plating layer disposed between the waveguide and the ceramic layer to prevent an inflow of a fluid into the electromagnetic wave sensor. A size of the ceramic layer plated with the thin metal plating layer may match a size of an internal space of the waveguide. The ceramic layer may include the dielectric having a permittivity proportional to a permittivity of a target to be sensed. The permittivity of the dielectric may be within a predetermined error range of a human body tissue permittivity. The fluid may include any one of a matching fluid and a gel-type semi-fluid to reduce reflection of the electromagnetic wave between a human body tissue which is a target to be sensed and the electromagnetic wave sensor. The thin metal plating layer may be disposed on an inner side of the waveguide, and the ceramic layer may be surrounded by the thin metal plating layer.

According to another aspect of the present invention, there is provided a multichannel sensing image processing method including arranging a plurality of electromagnetic wave sensors including at least one thin metal plating layer, calculating an electric field distribution corresponding to the electromagnetic wave sensors, and reconstructing a sensed image based on the calculated electric field distribution. The reconstructing may include setting the electric field distribution as an initial value for image restoration. The reconstructing may include changing an initial value corresponding to a single electromagnetic wave sensor to the electric field distribution. The reconstructing may include eliminating electromagnetic wave interference among the electromagnetic wave sensors. The electromagnetic wave sensors may be provided in a form of a structure in which a plurality of ceramic layers plated with the thin metal plating layer is arranged.

According to still another aspect of the present invention, there is provided a method of generating a multichannel electromagnetic wave sensor, the method including generating a ceramic layer including a dielectric to reduce a dielectric loss of an electromagnetic wave, plating the ceramic layer with a thin metal film, and arranging the ceramic layer. The ceramic layer may include the dielectric having a permittivity proportional to a permittivity of a target to be sensed by the multichannel electromagnetic wave sensor. The permittivity of the dielectric may be within a predetermined error range of a human body tissue permittivity. The method may further include calculating an electric field distribution corresponding to the arrangement of the ceramic layer, and setting the calculated electric field distribution as an initial value for image restoration.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1A is a perspective view illustrating a waveguide and an internal structure of the waveguide according to an embodiment of the present invention;

FIGS. 1B and 1C are cross-sectional views of a waveguide according to an embodiment of the present invention;

FIG. 2 is a flowchart illustrating a multichannel sensing image processing method according to an embodiment of the present invention;

FIG. 3 is a flowchart illustrating a method of generating a multichannel electromagnetic wave sensor according to an embodiment of the present invention; and

FIGS. 4A and 4B illustrate multichannel sensing arrangements according to embodiments of the present invention.

### DETAILED DESCRIPTION

Reference will now be made in detail to example embodiments of the present invention, examples of which are illustrated in the accompanying drawings, wherein like reference numerals refer to the like elements throughout. Example embodiments are described below to explain the present invention by referring to the accompanying drawings and the present invention is, however, not limited thereto or restricted thereby.

Unless otherwise defined, all terms including technical and scientific terms used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Terms used herein are defined to appropriately describe the example embodiments of the present invention and thus may be changed depending on a user, the intent of an operator, or a custom. Also, some specific terms used herein are selected by applicant(s) and such terms will be described in detail. Accordingly, the terms used herein must be defined based on the following overall description of this specification.

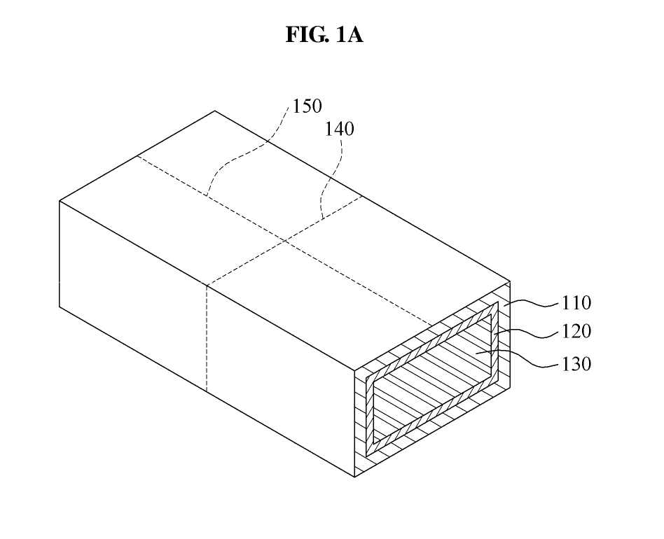

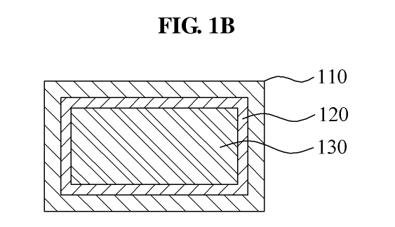

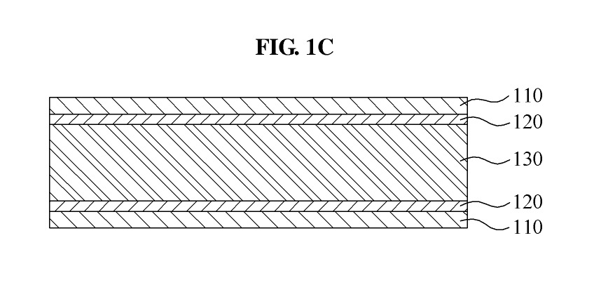

FIG. 1A is a perspective view illustrating a waveguide and an internal structure of the waveguide 110 according to an embodiment of the present invention.

Referring to FIG. 1A, the internal structure of the waveguide 110 includes a thin metal plating layer 120 and a ceramic layer 130. According to an embodiment, an electromagnetic wave sensor includes the thin metal plating layer 120 as the internal structure of the waveguide 110 to prevent an inflow of a fluid into the electromagnetic wave sensor and deterioration in a performance of the electromagnetic wave sensor. In an example, the thin metal plating layer 120 may be disposed on an inner wall of the waveguide 110, and the ceramic layer 130 may be surrounded by the thin metal plating layer 120. The electromagnetic wave sensor including the thin metal plating layer 120 may obtain a sensor gain in comparison to a general electromagnetic wave sensor.

The waveguide 110 functions as a passage through which an electromagnetic wave passes. The waveguide 110 may include a conductor. The electromagnetic wave may pass through the conductor. The electromagnetic wave may include information about a target to be sensed, hereinafter referred to as a sensing target. For example, the sensing target may be a human body tissue.

The thin metal plating layer 120 is disposed between a metal wall of the conductor and the ceramic layer 130. The thin metal plating layer 120 may prevent an inflow of a fluid into the electromagnetic wave sensor. The fluid may reduce reflection of an electromagnetic wave between the sensing target and the electromagnetic wave sensor. For example, the fluid may include any one of a matching fluid and a gel-type semi-fluid.

A general electromagnetic wave sensor may be generated using a method of inserting, in a waveguide, a ceramic layer manufactured to match a metal wall of the waveguide and tightly connecting to the waveguide. The general electromagnetic wave sensor may have a fine gap between the metal wall of the waveguide and the ceramic layer because the gap is inevitably generated due to a manufacturing tolerance. Such a fine gap may absorb a fluid used in a sensing process through a capillary phenomenon. The fluid may be a loss factor having a complex permittivity in the electromagnetic wave sensor. Further, current typically flows along an inner wall of the waveguide based on a characteristic of the waveguide and thus, a lossy fluid may deteriorate a performance of the electromagnetic wave sensor. Accordingly, a sensor gain may decrease and an electromagnetic field in the electromagnetic wave sensor may be disturbed and thus, a sensor reflection loss different from a designed value may occur.

According to an embodiment, the thin metal plating layer 120 of the electromagnetic wave sensor is plated on an outer surface of the ceramic layer 130. In such a case, an electromagnetic field in the ceramic layer 130 may preferentially make contact with the thin metal plating layer 120 and thereby, blocking a contact with a fluid. In an example, a thickness of the thin metal plating layer 120 may be designed to be proportional to a size of an internal space of the waveguide 110. In detail, a combined size of the thin metal plating layer 120 and the ceramic layer 130 may be designed to match the size of the internal space of the waveguide 110.

The ceramic layer 130 may be accommodated in the waveguide 110. The ceramic layer 130 may include a dielectric to reduce a dielectric loss of the electromagnetic wave. In an example, the ceramic layer 130 may include the dielectric having a permittivity proportional to a permittivity of a sensing target.

The ceramic layer 130 may include the dielectric having a permittivity within a range predetermined based on the permittivity of the sensing target for which the electromagnetic wave sensor is used. For example, the sensing target may be a human body tissue. The permittivity of the dielectric of the ceramic layer 120 may be within a predetermined error range of a human body tissue permittivity. The human body tissue may have a higher permittivity compared to other dielectrics. Thus, the electromagnetic wave sensor may include the ceramic layer 130 having the permittivity within the predetermined range in comparison to the human body tissue permittivity.

FIGS. 1B and 1C are cross-sectional views of the waveguide 110 according to an embodiment of the present invention.

Referring back to FIG. 1A, a cross section 140 and a cross section 150 are obtained from the internal structure of the waveguide 110. FIG. 1B illustrates the cross section 140, and FIG. 1C illustrates the cross section 150. Referring to FIG. 1B, based on an outermost contour, the waveguide 110, the thin metal plating layer 120, and the ceramic layer 130 are disposed. Current generally flows between the waveguide 110 and the thin metal plating layer 120 based on a characteristic of the waveguide 110. In a general electromagnetic wave sensor, fluids used to prevent reflection of an electromagnetic wave may permeate through a fine gap and generate a dielectric loss. The thin metal plating layer 120 may prevent such an inflow of the fluids. Referring to FIG. 1C, in a vertical structure, the waveguide 110, the thin metal plating layer 120, and the ceramic layer 130 are disposed, and the thin metal plating layer 120 and the waveguide 110 are disposed on and above the ceramic layer 130.

Despite an inflow of a fluid between an inner wall of the waveguide 110 and the thin metal plating layer 120, the inflow may not affect an electromagnetic field flow of the electromagnetic wave sensor. In a case that a fluid flows in through a coaxial feeder in the waveguide 110 to which an electromagnetic field is input or an opening in the waveguide 110 from which the electromagnetic field is emitted, and the electromagnetic field is excited, an equivalent circuit of an inner wall of the conductor of the waveguide 110 and the thin metal plating layer 120 may be substituted by a line through which a current flows smoothly. Also, an equivalent circuit of a fluid flowing in between lines may be substituted by a resistance connected in parallel between the existing parallel lines. A high impedance connected between the parallel lines may indicate the same electrical characteristics as an open circuit and thus, an impedance value of the fluid may be omitted.

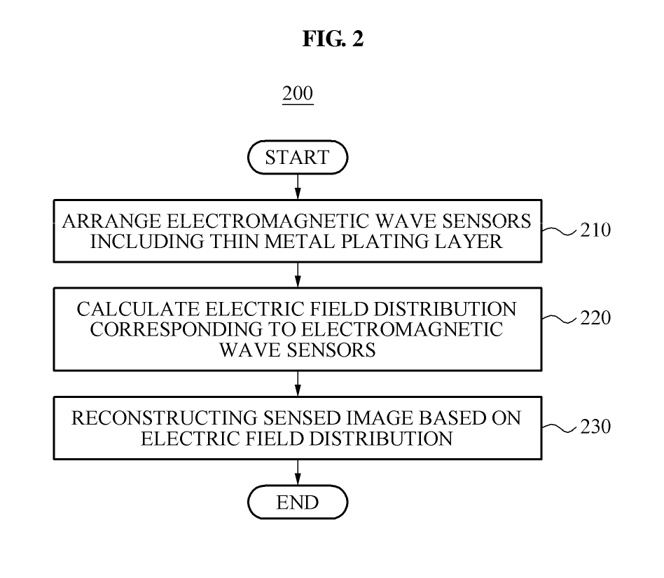

FIG. 2 is a flowchart illustrating a multichannel sensing image processing method 200 according to an embodiment of the present invention.

Referring to FIG. 2, the multichannel sensing image processing method 200 includes operation 210 of arranging a plurality of electromagnetic wave sensors including at least one thin metal plating layer, operation 220 of calculating an electric field distribution corresponding to the electromagnetic wave sensors, and operation 230 of reconstructing a sensed image based on the calculated electric field distribution. According to an embodiment, the electromagnetic wave sensors may enable a multichannel sensing arrangement. A dense arrangement of the electromagnetic wave sensors may change an existing electromagnetic wave radiation characteristic and generate a new electromagnetic wave radiation characteristic. Thus, a new image processing method may be necessary. In such a case, the multichannel sensing image processing method 200 may not affect a quality of an image.

In operation 210, the electromagnetic wave sensors including the thin metal plating layer are arranged. The electromagnetic wave sensors may be provided in a form of a structure in which a plurality of ceramic layers plated with the thin metal plating layer is arranged. In an example, the ceramic layers may be arranged in a horizontal direction. In another example, the ceramic layers may be arranged in a vertical direction. The ceramic layers may be arranged one-dimensionally. In still another example, the ceramic layers may be arranged in a planar direction including the horizontal arrangement and the vertical arrangement. In yet another example, the ceramic layers may be arranged in a circular form. The ceramic layers may be arranged two-dimensionally. In further another example, the ceramic layers may be arranged in a three-dimensional form surrounding a region to be measured. The three-dimensional form may include a spherical form.

Conventionally, to arrange a plurality of electromagnetic wave sensors, a conductor wall of a metal waveguide may be manufactured to be thin. As a greater number of electromagnetic wave sensors are arranged, a higher image quality may be obtained. However, when the conductor wall of the waveguide is processed to be thinner than a predetermined value, the waveguide may be bent or torn.

The electromagnetic wave sensors may be an electromagnetic wave sensor of which a thickness of the conductor wall of the waveguide is reduced by a thickness equal to a thickness of the thin metal plating layer being plated. The electromagnetic wave sensors may be an electromagnetic wave sensor of which the thin metal plating layer takes the place of the conductor wall of the waveguide.

In operation 220, the electric field distribution corresponding to the electromagnetic wave sensors is calculated. The electromagnetic wave sensors are arranged to perform multichannel sensing. Such a multichannel electromagnetic wave sensor may have an electromagnetic wave radiation characteristic different from an electromagnetic wave radiation characteristic of an existing single electromagnetic wave sensor. Thus, in operation 220, an electric field distribution newly generated based on a dense arrangement of the electromagnetic wave sensors is calculated.

In operation 230, the sensed image is reconstructed based on the calculated electric field distribution. Operation 230 may include setting the electric field distribution as an initial value for image restoration. In such a case, operation 230 may include changing, to the electric field distribution, a previously calculated initial value corresponding to a single electromagnetic wave sensor. Operation 230 may further include eliminating an electromagnetic wave interference among the electromagnetic wave sensors. Since the electric field distribution corresponding to the dense arrangement of the electromagnetic wave sensors is set as the initial value, a multichannel sensed image may be obtained without affecting a quality of a reconstructed image. The multichannel sensing image processing method 200 may further include processing a calculation for reconstructing the sensed image. In addition, the multichannel sensing image processing method 200 may further include outputting an image obtained through the calculation.

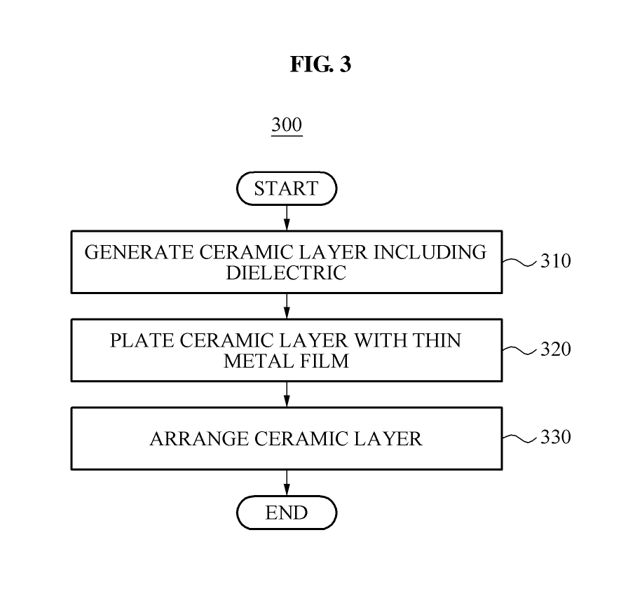

FIG. 3 is a flowchart illustrating a method 300 of generating a multichannel electromagnetic wave sensor according to an embodiment of the present invention.

Referring to FIG. 3, the method 300 includes operation 310 of generating a ceramic layer including a dielectric, operation 320 of plating the ceramic layer with a thin metal film, and operation 330 of arranging the ceramic layer. The method 300 may densely arrange a greater number of electromagnetic wave sensors, in comparison to conventional technology. For example, the multichannel electromagnetic wave sensor generated through the method 300 may be used for an electromagnetic wave imaging apparatus for medical purposes.

In operation 310, the ceramic layer including the dielectric is generated. The dielectric may have a permittivity to reduce a dielectric loss of an electromagnetic wave. The permittivity of the dielectric may be proportional to a permittivity of a sensing target. For example, the permittivity of the dielectric may be within a predetermined error range of a human body tissue permittivity. The electromagnetic wave may include information about the sensing target.

In operation 320, the ceramic layer is plated with the thin metal film. In an example, a thickness of an inner metal wall of a waveguide of a general electromagnetic sensor may be equal to a combined thickness of an inner metal wall of a waveguide and a thin metal plating layer of the electromagnetic wave sensor disclosed herein. That is, a thickness of the plated thin metal film may be substituted for the thickness of the inner metal wall of the waveguide. Operation 320 may further include generating the waveguide and generating the ceramic layer proportional to a size of an internal space of the waveguide. Operation 320 may include plating a portion in which the ceramic layer is in contact with the inner wall of the waveguide and combining the ceramic layer and the waveguide.

In operation 330, the ceramic layer is arranged. In an example, when substituting the inner metal wall of the waveguide with the thin metal plating layer, a number of electromagnetic wave sensors that may be arranged in a diagnostic imaging apparatus may increase. Thus, compared to conventional technology, a greater number of electromagnetic wave sensors may be densely arranged and thus, a sensed image with an improved accuracy may be obtained.

In operation 330, a plurality of ceramic layers generated in operation 320 is arranged. In an example, the ceramic layers may be arranged in a horizontal direction. In another example, the ceramic layers may be arranged in a vertical direction. In such examples, the ceramic layers may be arranged one-dimensionally. In still another example, the ceramic layers may be arranged in a planar direction including the horizontal arrangement and the vertical arrangement. In yet another example, the ceramic layers may be arranged in a circular form. In such examples, the ceramic layers may be arranged two-dimensionally. In further another example, the ceramic layers may be arranged in a three-dimensional form surrounding a region to be measured. The three-dimensional form may include a spherical form.

Operation 330 may further include calculating an electric field distribution corresponding to the arrangement of the ceramic layers, and setting the calculated electric field distribution as an initial value for image restoration. Thus, deterioration in a quality of an entire restored image may be prevented.

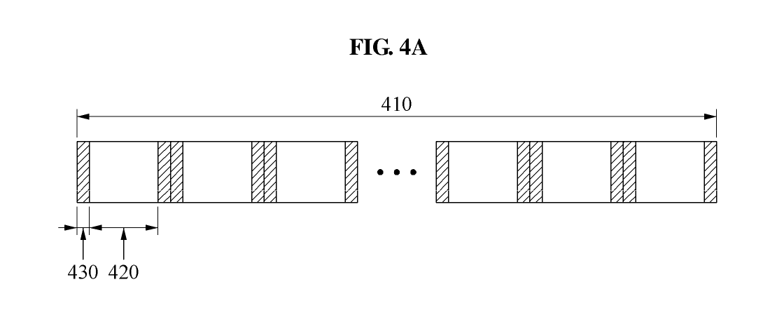

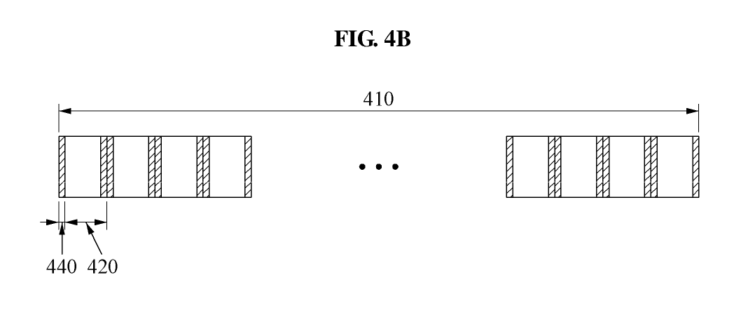

FIGS. 4A and 4B illustrate multichannel sensing arrangements according to embodiments of the present invention.

FIG. 4A illustrates an arrangement of a plurality of general electromagnetic wave sensors. Referring to FIG. 4A, a maximum length 410 of a designable arrangement, which is assumed to be a constant having a unit, a thickness 420 of a ceramic layer, and a thickness 430 of a metal wall of a waveguide are illustrated. Here, a number of electromagnetic wave sensors that may be included in a diagnostic imaging apparatus having the maximum length 410 may be obtained. A value obtained by dividing the maximum length 410 by a sum of the thickness 420 of the ceramic layer and a double value of the thickness 430 of the metal wall of the waveguide may correspond to a maximum number of insertable electromagnetic wave sensors.

FIG. 4B illustrates an arrangement of a plurality of electromagnetic wave sensors according to an embodiment of the present invention. Referring to FIG. 4B, a thickness 440 of a thin metal plating layer is illustrated. In an example, a plurality of electromagnetic wave sensors in which the thin metal plating layer is substituted for a metal wall structure of a waveguide by may be arranged. Here, a number of the electromagnetic wave sensors that may be included in a diagnostic imaging apparatus having a maximum length 410 may be obtained. A value obtained by dividing the maximum length 410 by a sum of a thickness 420 of a ceramic layer and a double value of a thickness 440 of the thin metal plating layer may correspond to a maximum number of insertable electromagnetic wave sensors.

Thus, the number of the electromagnetic wave sensors that may be included in the diagnostic imaging apparatus having the same length may increase by a thickness of the thin metal plating layer which is reduced with respect to a thickness of the metal wall of the waveguide. As described in the foregoing, reducing the thickness of the metal wall of the waveguide may incur a considerable amount of costs because the metal wall may be bent or torn during such a process. Thus, a greater number of electromagnetic wave sensors may be arranged in a diagnostic imaging apparatus of the same length by forming a multichannel sensing arrangement using the electromagnetic wave sensor disclosed herein. Therefore, a more accurate result value of sensing may be obtained.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An electromagnetic wave sensor, comprising:
a waveguide comprising a conductor to sense an electromagnetic wave;
a ceramic layer accommodated in the waveguide and comprising a dielectric to reduce a dielectric loss of the electromagnetic wave; and
a thin metal plating layer disposed between the waveguide and the ceramic layer to prevent an inflow of a fluid into the electromagnetic wave sensor.

2. The electromagnetic wave sensor of claim 1, wherein a size of the ceramic layer plated with the thin metal plating layer matches a size of an internal space of the waveguide.

3. The electromagnetic wave sensor of claim 1, wherein the ceramic layer comprises the dielectric having a permittivity proportional to a permittivity of a target to be sensed.

4. The electromagnetic wave sensor of claim 3, wherein the permittivity of the dielectric is within a predetermined error range of a human body tissue permittivity.

5. The electromagnetic wave sensor of claim 1, wherein the fluid comprises any one of a matching fluid and a gel-type semi-fluid to reduce reflection of the electromagnetic wave between a human body tissue which is a target to be sensed and the electromagnetic wave sensor.

6. The electromagnetic wave sensor of claim 1, wherein the thin metal plating layer is disposed on an inner wall of the waveguide, and the ceramic layer is surrounded by the thin metal plating layer.

7. A multichannel sensing image processing method, comprising:
arranging a plurality of electromagnetic wave sensors comprising at least one thin metal plating layer;
calculating an electric field distribution corresponding to the electromagnetic wave sensors; and
reconstructing a sensed image based on the calculated electric field distribution.

8. The method of claim 7, wherein the reconstructing comprises setting the electric field distribution as an initial value for image restoration.

9. The method of claim 8, wherein the reconstructing comprises changing an initial value corresponding to a single electromagnetic wave sensor to the electric field distribution.

10. The method of claim 7, wherein the reconstructing comprises eliminating electromagnetic wave interference among the electromagnetic wave sensors.

11. The method of claim 7, wherein the electromagnetic wave sensors are provided in a form of a structure in which a plurality of ceramic layers plated with the thin metal plating layer is arranged.

12. A method of generating a multichannel electromagnetic wave sensor, the method comprising:
generating a ceramic layer comprising a dielectric to reduce a dielectric loss of an electromagnetic wave;
plating the ceramic layer with a thin metal film; and
arranging the ceramic layer.

13. The method of claim 12, wherein the ceramic layer comprises the dielectric having a permittivity proportional to a permittivity of a target to be sensed by the multichannel electromagnetic wave sensor.

14. The method of claim 13, wherein the permittivity of the dielectric is within a predetermined error range of a human body tissue permittivity.

15. The method of claim 12, further comprising:
calculating an electric field distribution corresponding to the arrangement of the ceramic layer, and setting the calculated electric field distribution as an initial value for image restoration.
